(19) United States
(12) Patent Application Publication
(10) Pub. No.: US 2025/0352444 A1
(43) Pub. Date: Nov. 20, 2025

(54) Antimicrobial mixtures

(71) Applicant: Symrise AG, Holzminden (DE)

(72) Inventors: Christin KOCH, Detmold (DE); Benoit JOIN, Holzminden (DE); Arnold MACHINEK, Holzminden (DE); Tim SCHMITTER, Einbeck (DE); Jessica GRIEGER; Lisa GARBE, Holzminden (DE); Manuel PESARO, Romanel-sur-Lausanne (CH)

(21) Appl. No.: 18/865,901

(22) PCT Filed: May 15, 2023
(86) PCT No.: PCT/EP2023/062917

§ 371 Date: Nov. 14, 2024

(30) Foreign Application Priority Data
May 18, 2022 (WO) PCT/EP2022/063437

(51) Int. Cl. A61K 8/34; A61Q 11/00

(52) U.S. Cl. CPC A61K 8/347; A61Q 11/00; A61K 2800/92

(57) ABSTRACT

Suggested is an anti-microbial mixture comprising or (essentially) consisting of: (a) at least one flavoring agent with unsaturated six-membered ring structure, and (b) at least one physiological cooling agent.

### AREA OF INVENTION

The present invention refers to the area of oral care and proposes new compositions for fighting plaque and biofilms on teeth.

### BACKGROUND OF THE INVENTION

Nowadays the treatment of oral diseases, like caries or periodontitis is focused on preventive behavior. Thereby the reduction of plaque/biofilm and harm associated bacteria takes the center stage. The principal method is the mechanical removal of plaque and the use of toothpaste or mouthwashes that contain active ingredients that are known to control specific bacteria found in dental plaque. One of these well-known antimicrobial active ingredients is Carvacrol.

Looking at the overall biofilm the complexity of a naturally formed biofilm is a huge challenge when it comes to the development of a suitable test systems. Thus, applied solutions with specific anti-biofilm properties are hardly known and rarely used.

### RELEVANT PRIOR ART

EP 1830926 B1 (COLGATE) refers to a mixture that is efficacious as an antibacterial, antiplaque, anti-inflammatory, anti-oxidant, and anti-halitosis oral composition. The active ingredient has one or more active compounds from an extract of oregano, such as thymol, carvacrol and rosmarinic acid. The oral composition can be in the form of a mouth rinse; a dentifrice, including toothpaste, gels, powders; animal products; a film; or confectioneries, such as lozenges, and the like.

WO 2006 053458 A1 (GIVAUDAN) relates to bactericidal formulations that come into contact with human body, particularly wash formulations including liquid soaps, comprising perfume ingredients active against gram-negative bacteria, hydrotropes and surfactants. Such compositions contact the target site, for example the skin surface, only for a short time, usually not longer than 30 seconds.

WO 2008 068683 A1 (FIRMENICH) concerns a flavoring composition for use in an oral care product, confectionary product or beverage. The composition includes an antimicrobial key and optionally at least one flavoring ingredient of current use, wherein the antimicrobial key comprises 3,4-dimethylphenol together with one or more antimicrobial flavor ingredients each having a minimum inhibitory concentration of 1000 parts per million or less, against two or more strains selected from Fusobacterium nucleatum, Fusobacterium sp., Porphyromonas gingivalis, Prevotella intermedia, Klebsiella pneumoniae, Veillonella alcalescens, Bacteroides melaninogenicus/forsythus, Selenomonas sputagena, Porphyromonas endodontalis, Prevotella melaninogenica and Streptococcus mutans.

WO 2011 055708 A1 (LION) refers to a liquid composition for the oral cavity containing a nonionic surfactant, a wetting agent selected from among glycerin, propylene glycol, polyethylene glycols and sorbitol, 1-menthol, a para-hydroxybenzoate and a fragrance material selected from among 3-octanol, 3-octyl acetate, 3-octanone and fenchone.

DE 10 2014 221451 A1 (HENKEL) refers to oral and dental care and cleaning products, which-based on their weight—from 1 to 60 weight percent of at least one humectant selected from the group of sorbitol, glycerol or mixtures thereof, from 0.1 to 5 weight percent of at least one cooling agent from the group of menthol, menthyl lactate or mixtures thereof and 0.1 to 5 weight percent of an oily extract from the leaves of Rumex acetosa included, which show an improved cleaning performance and prevention of new plaque and also have a prolonged antibacterial effect.

FR 3105732 A1 (EZAL) relates to a composition comprising Thymol or an essential oil comprising thymol; carvacrol or an essential oil comprising carvacrol, cineol or an essential oil comprising cineol, Eugenol or an essential oil comprising Eugenol, limonene, or an essential oil, comprising limonene and an aqueous gelling agent. It also relates to the use of this composition as oral hygiene product and as a medicament, in particular for the treatment and/or prevention of an oral pathology and for preventing and/or reducing the formation of dental plaque.

U.S. Pat. No. 5,346,615 A (COLGATE) claims an antiplaque oral composition, such as a toothpaste, gel dentifrice, tooth powder, mouthrinse or mouthwash, tooth hardener, anticalculus composition, gum or lozenge, comprises triclosan as an antiplaque component, the antiplaque action of which is unexpectedly and beneficially increased by the presence in the oral composition of a phenolic flavoring agent, such as eucalpytol, thymol, methyl salicylate, menthol, phenol, halogenated derivatives thereof, and mixtures thereof.

U.S. Pat. No. 5,945,087 A (PFIZER) discloses oral rinse and dentifrice compositions, comprising a phenolic selected from the group consisting of menthol, eucalyptol, methyl salicylate, thymol, triclosan, and mixtures thereof; and a cyclodextrin. The compositions are useful in retarding the development of plaque, treating gingivitis, and in treating the presence of micro-organisms in the oral cavity.

US 2009 163530 AA (LEUNG) claims edible films that include pullulan and antimicrobially effective amounts of the essential oils thymol, methyl salicylate, eucalyptol and menthol. The edible films are effective at killing the plaque-producing germs that cause dental plaque, gingivitis and bad breath.

US 2012 014884 AA (COLGATE) discloses novel uses of specific menthol-derivative compounds for methods of providing oral and systemic health care benefits, and methods of up-regulating or down-regulating periodontal disease metabolites.

JP 2014 043441 A2 (SUNSTAR) provide a composition for oral cavity and a candida biofilm formation inhibitor containing geraniol and menthol.

### OBJECT OF THE INVENTION

Therefore, it has been the object of the present invention providing new actives or active compositions capable of fighting biofilms, particularly plaque in the oral cavity, which are simultaneously
highly efficient even at low concentrations against all or at least most types of microorganisms causing such biofilms;
mild to mucous membranes;
exhibit a pleasant taste of freshness; and
improve antimicrobial activity.

### BRIEF DESCRIPTION OF THE INVENTION

A first object of the present invention refers to an antimicrobial mixture comprising or (essentially) consisting of:
(a) at least one flavoring agent, and
(b) at least one physiological cooling agent,
wherein said flavoring agent is selected from the group consisting of unsaturated 6-membered ring structures following formula (I) to (IX)

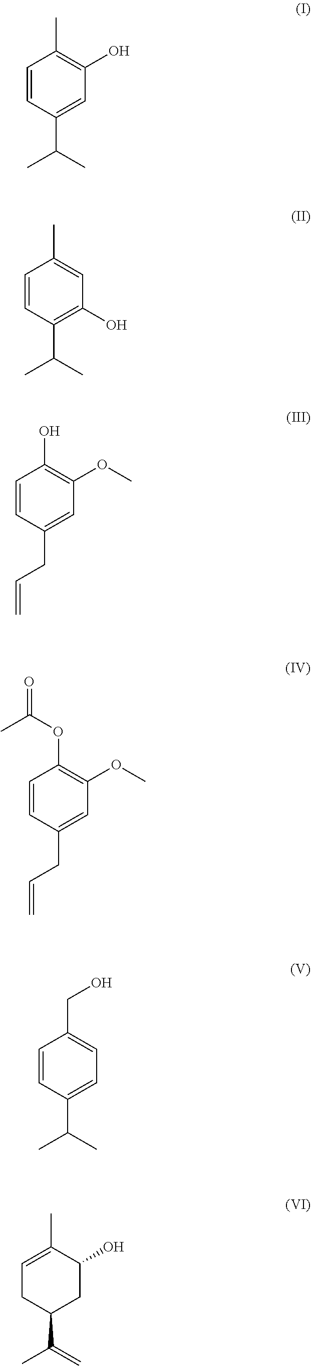

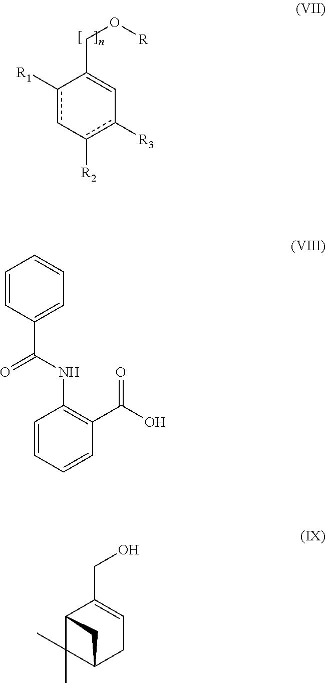

wherein R, R1, R2 and R3 independently from each other stand for hydrogen, C₁-C₃ alkyl, or C₃ alkenyl, C₁-C₃ alkoxy or acetate and n represent either zero or 1.

Surprisingly new synergistic efficacy in fighting micro-organisms causing biofilms in the oral cavity was observed, when combining the above referenced flavoring agents in general, and carvacrol in particular with physiological cooling agents. The anti-microbial efficacy against decisive bacteria strains occurring in human saliva can be improved by this synergistic effect. The new mixtures are particularly active against bacterial strains selected from the group consisting of P. gingivalis, F. nucleatum, P. intermedia and/or S. mutans. The products are mild to mucous membranes and provide a fresh and pleasant taste.

Synergistic effects were also observed with natural oils containing above referenced flavoring agents in general, and carvacrol in particular with physiological cooling agents.

### Flavoring Agents

In a preferred embodiment said unsaturated six-membered ring structures forming component (a) are selected from the group consisting of carvacrol, thymol, eugenol eugenyl acetate, dianthramide B, myrtenol, cumene alcohol and mixtures thereof and essential oils comprising at least one of said structures, such as for example oregano oil, thyme oil or majoran oil.

### Carvacrol, or Cymophenol

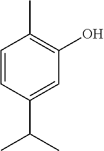

which is the preferred flavoring agent, is a monoterpenoid phenol having the characteristic warm odor of oregano. It can be produced synthetically or extracted from several natural sources. Carvacrol is present in the essential oil of oregano, oil of thyme, oil obtained from pepperwort, and wild bergamot. The essential oil of oregano and thyme subspecies contain between 5% and 75% of carvacrol, while Satureja (savory) subspecies have a content between 1% and 45%. Origanum majorana (marjoram) and Dittany of Crete are rich in carvacrol, 50% and 60-80% respectively. It is also found in tequila and Lippia graveolens (Mexican oregano) in the verbena family. Carvacrol is characterized by a strong disinfectant, fungicidal and bactericidal effect and is used in mouthwashes, toothpaste and in alcoholic solution for skin disinfection because of its pleasant taste (cf. Heinz Lüllmann, Klaus Mohr, Lutz Hein: Pharmakologie und Toxikologie, Georg Thieme, Stuttgart, 2010, p. 451.)

Thymol (also known as 2-isopropyl-5-methylphenol, IPMP),

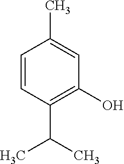

is a natural monoterpenoid phenol derivative of p-Cymene, isomeric with carvacrol, found in oil of thyme, origanum and majoram, ajwain, and various other plants as a white crystalline substance of a pleasant aromatic odor and strong antiseptic properties. Thymol also provides the distinctive, strong flavor of the culinary herb thyme, also produced from T. vulgaris.

### Eugenol

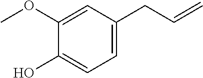

is an allyl chain-substituted guaiacol. It is a colorless to pale yellow, aromatic oily liquid extracted from certain essential oils especially from clove, nutmeg, cinnamon, basil and bay leaf. It is present in concentrations of 80-90% in clove bud oil and at 82-88% in clove leaf oil. Eugenol has a pleasant, spicy, clove-like scent. The name is derived from Eugenia caryophyllata, the former Linnean nomenclature term for cloves. The currently accepted name is Syzygium aromaticum.

### Eugenyl Acetate Also Known as Acetyl Eugenol

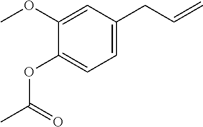

is a phenylpropanoid compound found in cloves. It is the second in abundance to the related compound eugenol in certain extract preparations. Like eugenol, it is found in several plants such as Acacia nilotica and Piper betle and has similar antibacterial and antifungal properties on C. albicans and S. mutans. It inhibits aggregation of platelets and has partial agonistic activity on AhR.

### Myrtenol

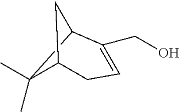

is a bio-active isolate of Taxus

### Physiological Cooling Agents

Said physiological cooling agents, forming component (b) are added for both, providing a cooling sensation and increasing the antimicrobial activity of the composition. They are preferably selected from the group formed by the species depicted in the following table (including their optical isomers and racemates):

| Component | FEMA/GRAS | Trademark | Supplier |
|---|---|---|---|
| (1R,2S,5R)-5-methyl-2-(propan-2-yl)-N-[2- | 4549 | Evercool 190 | Givaudan |
| (pyridine-2-yl)ethyl]cycloheaxecarboxamide | | | |
| Menthol | 2665 | | |
| L-Menthone glyceryl ketal | 3807 | Frescolat° MGA | Symrise AG |
| D,L-Menthone glyceryl ketal | 3808 | Frescolat° MGA | Symrise AG |
| Menthoxy-1,2-propandiol | 3784 | Cooling Agent 10 | |
| Menthoxy-2-methyl-1,2-propanediol | 3849 | | |
| Menthyl ethylene glycol carbonate | 3805 | Frescolat° MGC | Symrise AG |
| Menthyl formiate | 4509 | | |
| L-MonoMenthyl glutarate | 4006 | | |
| Menthyl hydroxy isobutyrate | 4308 | | |
| Menthyl isobutyrate | | | |
| L-Menthyl lactate | 3748 | Frescolat° ML | Symrise AG |
| Menthyl malonate | | | |
| Menthyl N-ethyl oxamate | | | |
| (−)-Menthyl propylene glycol carbonate | 3806 | Frescolat° MPC | Symrise AG |
| DL-Menthyl propylene glycol carbonate | 3992 | Frescolat° MPC | Symrise AG |
| Menthyl pyroglutamate | | | |
| Menthyl-(2-methoxy)acetate | | | |
| Menthyl-(2-methoxyethoxy)acetate | | | |
| Mono-Menthyl succinate | 3810 | | |
| O-Menthyl succinic acid ester amide | | | |
| O-Menthyl succinic acid ester-NN- | 4230 | | |
| (dimethyl)amide | | | |
| Menthane carboxylic acid-N-(4-cyanomethyl- | 4496 | Evercool 180 | |
| phenyl)amide | | | |
| Menthane carboxylic acid-N-(2-(pyridine-2- | 4549 | | |
| yl)ethyl)amide | | | |
| Menthane carboxylic acid-N-ethylamide (WS-3) | 3455 | | |
| (WS-4) | | | |
| N^{α}-(menthane carbonyl) glycinic ethylester (WS- | 4309 | | |
| 5) | | | |
| (1R,2S,5R)-N-(4-Methoxyphenyl)-5-methyl-2-(1- | 4681 | | |
| isopropyl)cyclohexane-carboxamide (WS-12) | | | |
| (WS-14) | | | |
| 2,3-dimethyl-2-(2-propyl)-butyric acid-N- | 3804 | | |
| methylamide (WS23) | | | |
| 3-(−)-Menthoxy)propane-1,2-diol | 3784 | | |
| Isopulegol acetate | 2965 | | |
| p-Menthane-3,8-diol | 4053 | | |
| Cubebol | 4497 | | |
| 3-Methyl-2(1-pyrrolidinyl)-2-cyclopentene-1- | | | |
| one) | | | |
| Tetrahydropyrimidine-2-one | | | |
| [(1R,2,5R)-2-isopropyl-5-methyl-cyclohexyl] 2- | | X Cool | Symrise AG |
| (ethylamino)-2-oxo-acetate | | | |
| 2-(p-Tolyloxy)-N-(1H-pyrazol-5-yl)-N- | 4809 | Freezestorm | |
| ((thiophen-2-yl)methyl)acetamide | | | |
| (E)-3-benzo[1,3]dioxol-5-yl-N,N-diphenyl-2- | 4788 | | |
| propenamide | | | |
| (E)-3-(1,3-benzodio-5-yl)-N-phenyl-N- | 5002 | | |
| tetrahydrofuran-3-yl-propo-2-enamide | | | |
| N-ethyl-2,2-diisopropylbutanamide (WS 27) | 4557 | | |
| 2-(4-Ethylphenoxy-N-(1H-pyrazol-5-yl)-N- | 4880 | | |
| ((thiophen-2-yl)methyl)acetamide | | | |
| N-(3-hydroxy-4-methoxyphenyl)-2-isopropyl- | 4881 | | |
| 5,5-dimethylcyclohexane-1-carboxamide | | | |
| N-(4-(cyanomethyl)phenyl)-2-isopropyl-5,5- | 4882 | Evercool 180 | |
| dimethylcyclohexane-1-carboxamide | | | |
| N-(2-hydroxy-2-phenylethyl)-2-isopropyl-5,5- | 4896 | | |
| dimethylcyclohexane-1-carboxamide | | | |
| 2-Methyl-1-(2-(5-(p-tolyl)-1H-imidazol-2- | 4970 | Evercool Blast | |
| yl)butan-1-one | | | |

A first important representative of the substances forming component (b) is monomenthyl succinate, which as a substance was patented as early as 1963 by Brown & Williamson Tobacco Corp. (U.S. Pat. No. 3,111,127) and as a refrigerant is the subject of property rights U.S. Pat. Nos. 5,725,865 and 5,843,466 (V. Mane Fils). Both the succinate and the analogous monomenthyl glutarate are important representatives of monomenthyl esters based on di- and polycarboxylic acids:

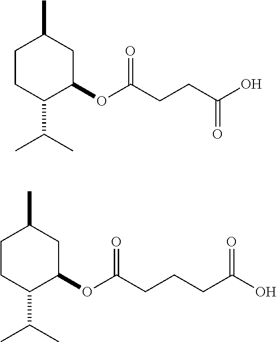

Examples of applications of these substances can be found, for example, in the printed documents WO 2003 043431 (Unilever) or EP 1332772 A1 (IFF).

The next important group of menthol compounds preferred in the sense of the invention comprises carbonate esters of menthol and polyols, such as glycols, glycerol or carbohydrates, such as menthol ethylene glycol carbonates, menthol propylene glycol carbonates, menthol 2-methyl-1,2-propanediol carbonates or the corresponding sugar derivatives:

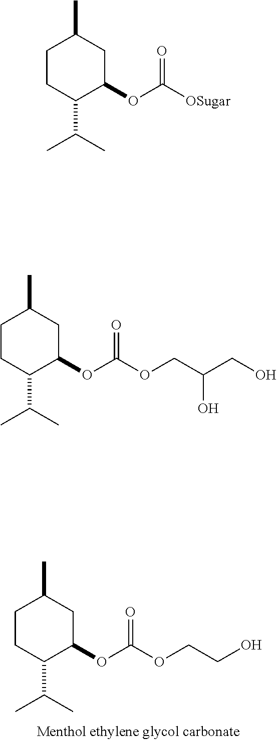

The use of such substances as a cooling agent for cigarettes is, for example, the subject of the 1968 publication U.S. Pat. No. 3,419,543 (Mold et al.); their use as a physiological cooling agent is claimed in DE 4226043 A1 (H&R).

In the sense of the invention, the preferred menthol compounds are menthone glyceryl acetal, menthone glyceryl ketal and menthyl lactate, wherein menthyl lactate is most preferred.

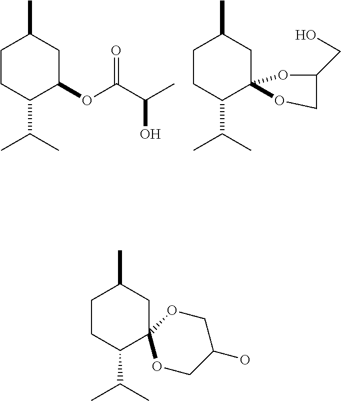

The former structure is obtained by esterification of lactic acid with menthol, the latter by acetylation of menthone with glycerol (cf. DE 2608226 A1, H&R). This group of compounds also includes 3-(I-menthoxy)-1,2,propanediol, also known as Cooling Agent 10 (U.S. Pat. No. 6,328,982, TIC), and 3-(I-menthoxy)-2-methyl-1,2,propanediol, which has an additional methyl group.

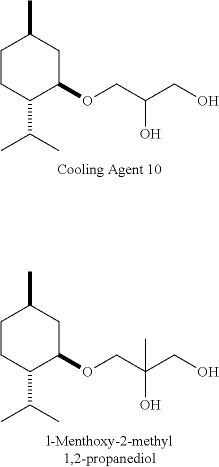 [SMILES: CC(C)[C@@H]1CC[C@@H](C)C[C@H]1OCC(O)CO ; CC(C)[C@@H]1CC[C@@H](C)C[C@H]1OCC(C)(O)CO]

For example, 3-(I-menthoxy)-1,2,propanediol is prepared starting from menthol according to the following scheme (see U.S. Pat. No. 4,459,425, Takasago):

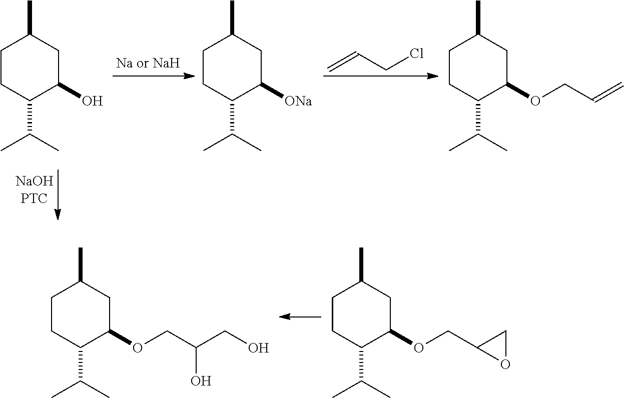

Alternative routes in which menthol is reacted with epichlorohydrin in the first step are described in U.S. Pat. Nos. 6,407,293 and 6,515,188 (Takasago). The following is an overview of preferred menthol compounds characterized by CO bonding:

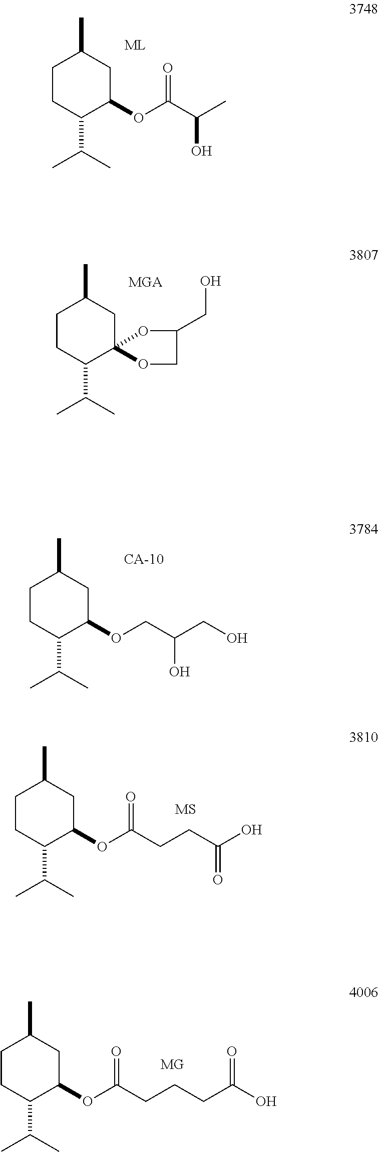

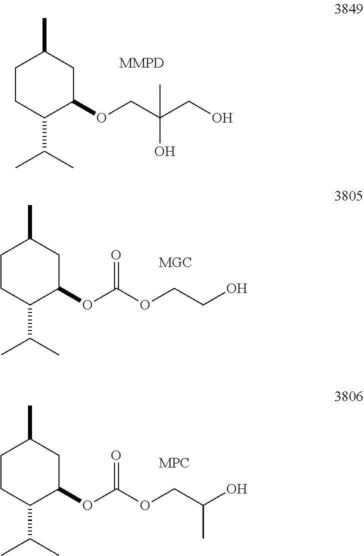

Among these substances, menthone glyceryl acetal/ketal and menthyl lactate as well as menthol ethylene glycol carbonate and menthol propylene glycol carbonate.

In the 1970s, menthol compounds were developed for the first time which have a C—C bond in the 3-position and of which a number of representatives can also be used in the sense of the invention. These substances are generally referred to as WS types. The basic body is a menthol derivative in which the hydroxyl group is replaced by a carboxyl group (WS-1). All other WS types are derived from this structure, such as the species WS-3, WS-4, WS-5, WS-12, WS-14 and WS-30, which are also preferred in the sense of the invention. The 10 following two diagrams show the synthesis routes:

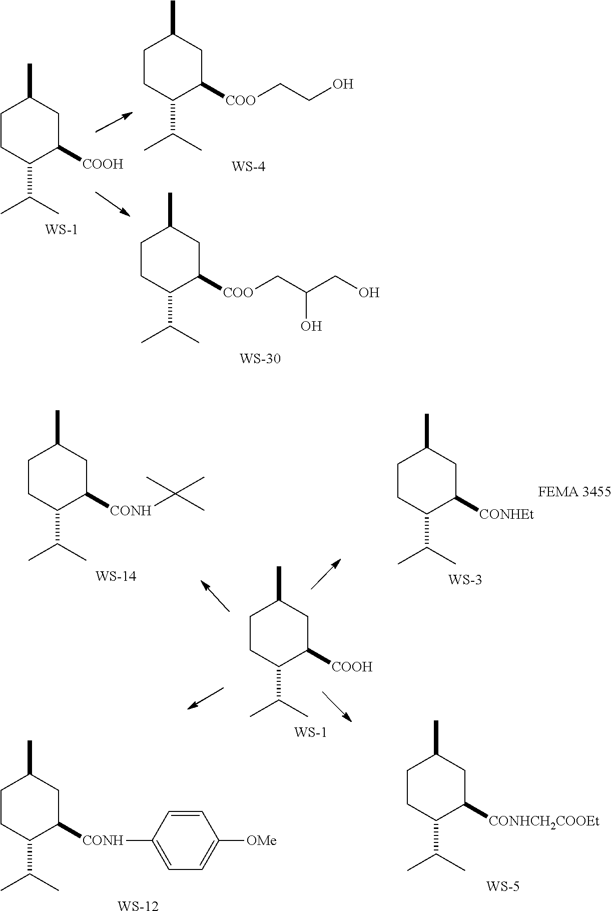

The esters derived from WS-1 are described, for example, in U.S. Pat. No. 4,157,384, and the corresponding N-substituted amides in J. Soc. Cosmet. Chem. pp. 185-200 (1978).

The preferred physiological cooling agents encompass menthyl lactate, menthyl propylene glycol carbonate, menthone glyceryl acetal/ketal and 3-(−)-Menthoxy) propane-1,2-diol.

In another preferred embodiment components (a) and (b) can be present in ratios by weight of from about 1:99 to about 99:1, preferably 10:90 to about 90:10, preferably from about 20:80 to about 80:20 and more preferred from about 40:60 to about 60:40.

### Solvents

In another preferred embodiment the mixtures according to the present invention may include solvents such as for example water, ethanol, 1,2-propylene glycol, 1,3-propylene glycol, triacetine, vegetable oil, triglycerides or glycerol and triethyl citrate or mixtures thereof. In case solvents are present, their amount ranges typically from about 5 to about 95 wt.-percent, preferably from about 10 to about 75 wt.-percent and more preferably from about to about 50 wt.-percent.

In case aromatic solvents are present, their amount ranges typically from about 5 to about 95 wt.-percent, preferably from about 10 to 90 wt.-percent and more preferable from to 80 wt.-percent.

### Aroma Compounds

In another preferred embodiment the mixtures according to the present invention may include aroma compounds, for example selected from Anethol, Acetanisol, Acetaldehyde, Acetylmethylcarbinol, 2-Acetylpyrazine, 2-Acetylpyridine, 2-Acethylthiazoline, 2-Acethylthiazole, Allylcapronate, alpha-Amylcinnamicaldehyde, para-Anisaldehyde, Anisalcohol, Dimethylisopropyldithiazine, Benzaldehyde, Benzylacetate, l-Borneol, Butiric acid, Butylacetate, 3-Butylidenphtalide, Capric acid, l-Carvone, d-Carvone, Carvomenthone, cisCarvylacetate, Caryophyllene, 1,8-Cineol, 1,4-Cineol, Cinnamylacetate, Citral, Citronellal, Citronellol, Citronellylacetate, Cuminaldehyde, Cyclopentadecanolide, alpha-Damascone, betaDamascone, alpha-Damascenone, beta-Damascenone, delta-Decalactone, gammaDecalactone, Dehydromenthofurolactone, Dihydromenthofurolactone, 2,3-Diethylpyrazine, Dihydroanethol, Dihydrocarvone, Dihydrocoumarine, beta-Dihydroionone, Dimethylanthranilate, Dimethylsulfide, Dimethylpyrazine, Sotolon, Diphenyloxide, Divanilline, 2,4-Decadienal, delta-Dodecalacton, gamma-Dodecalactone, Acetic acid, Ethylacetate, Ethylbutyrate, 2-Ethylmethylbutyrate, Ethylcapronate, Ethylcaprylate, Ethylcinnamate, Ethylisobutyrate, Ethylvanilline, Ethyllactate, Ethylmaltol, Ethylmethylthiopropionate, 4-Ethylphenol, Ethylisovalerianate, Eugenol, Fenchol, Furaneol, Filbertone, Frambinone, Frambinonmethylether, Furfurylthiol, Undecatriene, Geraniol, Geranylacetate, Geranylisobutyrate, Guaiacol, Heliotropine, 2-Heptanone, 4-Z-Heptenal, gamma-Hexalactone, gamma-Heptalactone, 3-Z-Hexenol, 2-E-Hexenol, Hexanol, Hexylacetate, 3-Z-Hexenylacetate, 2-E-Hexenylacetate, alpha-Hexylcinnamicaldehyde, 3-Z-Hexenylcapronate, Hotrienol, Indol, alpha-Irone, alphalonone, beta-Ionone, Isoamylacetate, Isoamylbutyrate, Isoamylisovaerianate, Isobutylacetate, Isobutylthiazol, Isobutyraldehyde, Isovaleraldehyde, Isoeugenol, Isomenthone, Isopropylmethoxypyrazine, Isobutylmethoxypyrazine, 2,4-Isopropylmethylpyrazine, Isopulegol, Jasminlactone, cis-Jasmone, Campher, Ketoisophorone, Cresol, d-Limonene, l-Linalool, d-Linalool, Linalylacetate, Linalooloxide, Maltol, Methylcyclopentenolone, L-Menthone, D-Menthone, LMenthol, D-Menthol, Neo Menthol, L-Menthylacetate, D-Menthylacetate, Massoilactone, Melonal, 1,8-Menthenthiol, 1,8-Epithiomenthane, 8,3-Thiomenthanone, Menthofurolactone, Menthadienylacetate, 2,3-Methoxymethylpyrazine, Methylanthranilate, Methylsalicylate, Thymol, Methylbutyrate, 2-Methylbutylacetate, Methylcinnamate, 2,3-Methylfuranthiol, 2,3-Methyltetrahydrofuranthiol, Methyljasmonate, Methyldihydrojasmonate, Methylthiobutyrate, 1,3-Methylthiohexylacetate, 1,3-Methylthiohexanol, Methional, Myrtenal, Naringin, Neral, Nerol, Nerylacetate, gamma-Nonalactone, delta-Nonalactone, 2-E-Nonenal, 6-Z-Nonenal, 6-Z-Nonenol, Nootkatone, Dihydronootkatone, 1,3-Octenol, gamma-Octalactone, deltaOctalactone, Pelitorine, 1,3-Pentenone, Pentylacetate, Phenylacetaldehyde, Phenylethylalcohol, Phenylethylacetate, Piperitanat, Prenylthiol, Prenylthioacetate, Rosenoxide, Rubenamine, Rubescenamine, Sabinenhydrate, Skatole, Styrolylacetate, Terpineol, 4-Terpinenol, 1,3-Thiohexanol, 1,3-Thiohexylacetate, 2,4,4-Thiopentanone, Trimethylpyrazine, delta-Undecalactone, gamma-Undecalactone, 2,4-Decadienal, 2,4-Nonadienal, 2,6-Nonadienal, 2,4-Undecadienal, Vanilline, Vinylguaiacol, Whiskylactone, Cinnamicaldehyde, Cinnamicalcohol, Diallyldisulfide, Allylisothiocyanate, Hexanal, E-2-Hexenal, Octanal, Decanal, Tridecatrienal, 12-Methyltridecanal, alpha-Pinene, beta-Pinene, Piperitone, as well as their mixtures.

### Oral Compositions

Another object of the present invention is directed to a preferably non-therapeutic oral and/or oral care composition, containing the antimicrobial mixtures preferably in amounts of from about 0.001 to about 1.5 wt.-percent, more preferably from about 0.01 to about 0.8 wt.-percent and most preferred from about 0.02 to about 0.3 wt.-percent-calculated on the composition.

Typical examples for suitable oral compositions encompass, toothpastes, mouth washes, (dental) chewing gums, (hard boiled) candies, and compressed tablets. The manufacture and composition of said oral compositions are described as follows:

### Toothpastes and Mouthwashes

Toothpastes or tooth creams are generally understood to be paste-like preparations of water, thickeners, humectants, abrasives or polishes, surfactants, sweeteners, flavorings, deodorizing agents and agents active against oral and dental diseases. In toothpastes according to the invention, any of the usual polishes may be used, such as chalk, dicalcium phosphate, insoluble sodium metaphosphate, aluminium silicates, calcium pyrophosphate, finely particulate synthetic resins, silicas, aluminium oxide and aluminium oxide trihydrate. Particularly suitable polishes for toothpastes according to the invention are finely particulate xerogel silicas, hydrogel silicas, precipitated silicas, aluminium oxide trihydrate and finely particulate .alpha-alumina, or mixtures of these polishes. Such polishes are preferably used in quantities of from about 15 to 40% by weight of the toothpaste. Preferred humectants used for toothpastes according to the invention include low molecular weight polyethylene glycols, glycerol, sorbitol or mixtures thereof in quantities of up to about 50% by weight of the toothpaste. Among the known thickeners for use with toothpastes according to the invention, particularly preferred are the thickening, finely particulate gel silicas and nonionic hydrocolloids, such as hydroxy ethyl cellulose, hydroxy propyl guar, hydroxy ethyl starch, polyvinyl pyrrolidone, high molecular weight polyethylene glycol and vegetable gums, such as tragacanth, agaragar, carrageen moss, gum arabic and xanthan gum. The desired flavor and aroma for preparations in accordance with the invention may be obtained by adding the components (a) and/or (b) and optionally also (c). It is also advantageous adding caries inhibitors to the oral preparations in the form of, for example, alkali fluorides, alkali monofluorophosphates or alkali salts of organophosphonic acids. In addition, the oral preparations according to the invention may contain other standard auxiliaries, such as dyes, preservatives and opacifiers, for example titanium dioxide. For mouthwashes, the oral compositions according to the invention may readily be combined with aqueous-alcoholic solutions containing different amounts of ethereal oils, emulsifiers, astringent and toning drug extracts, caries-inhibiting additives and flavor correctants.

### Chewing Gums

Chewing gums typically consist of a water-insoluble vase component, a water-soluble component and additives providing for example a specific flavour.

The water-insoluble base, which is also known as the “gum base”, typically comprises natural or synthetic elastomers, resins, fats and oils, plasticizers, fillers, softeners, dyes and optionally waxes. The base normally makes up 5 to 95% by weight, preferably 10 to 50% by weight and more particularly 20 to 35% by weight of the composition as a whole. In one typical embodiment of the invention, the base consists of 20 to 60% by weight synthetic elastomers, 0 to 30% by weight natural elastomers, 5 to 55% by weight plasticizers, 4 to 35% by weight fillers, 5 to 35% by weight softeners and small amounts of additives, such as dyes, antioxidants and the like, with the proviso that they are soluble in water at best in small quantities.

Suitable synthetic elastomers are, for example, polyisobutylenes with average molecular weights (as measured by GPC) of 10,000 to 100,000 and preferably 50,000 to 80,000, isobutylene/isoprene copolymers (“butyl elastomers”), styrene/butadiene copolymers (styrene: butadiene ratio, for example, 1:3 to 3:1), polyvinyl acetates with average molecular weights (as measured by GPC) of 2,000 to 90,000 and preferably 10,000 to 65,000, polyisoprenes, polyethylenes, vinyl acetate/vinyl laurate copolymers and mixtures thereof. Examples of suitable natural elastomers are rubbers, such as for example smoked or liquid latex or guayuls, and natural gums, such as jelutong, lechi caspi, perillo, sorva, massaranduba balata, massaranduba chocolate, nispero, rosindinba, chicle, gutta hang kang and mixtures thereof. The choice of the synthetic and natural elastomers and their mixing ratios essentially depends on whether or not bubbles are to be produced with the chewing gums (bubble gums). Elastomer mixtures containing jelutong, chicle, sorva and massaranduba are preferably used.

In most cases, the elastomers are too hard or lack plasticity for satisfactory processing, so that it has been found to be of advantage to use special plasticizers which, of course, must also satisfy in particular all requirements relating to acceptability as food additives. In this respect, suitable plasticizers are, above all, esters of resin acids, for example esters of lower aliphatic alcohols or polyols with completely or partly hydrogenated, monomeric or oligomeric resin acids. In particular, the methyl, glycerol or pentaerythritol esters or mixtures thereof are used for this purpose. Alternatively, terpene resins, which may be derived from alpha-pinene, beta-pinene, delta-limonene or mixtures thereof, could also be used.

Suitable fillers or texturizers are magnesium or calcium carbonate, ground pumice stone, silicates, especially magnesium or aluminium silicates, clays, aluminium oxides, talcum, titanium dioxide, mono-, di- and tricalcium phosphate and cellulose polymers.

Suitable softeners or emulsifiers are tallow, hydrogenated tallow, hydrogenated or partly hydrogenated vegetable oils, cocoa butter, partial glycerides, lecithin, triacetin and saturated or unsaturated fatty acids containing 6 to 22 and preferably 12 to 18 carbon atoms and mixtures thereof.

Suitable dyes and whiteners are, for example, the FD&C types, plant and fruit extracts permitted for coloring foods and titanium dioxide. The gum bases may also contain waxes or may be wax-free

In addition to the water-insoluble gum base, chewing gum preparations regularly contain a water-soluble component which is formed, for example, by softeners, sweeteners, fillers, flavours, flavour enhancers, emulsifiers, dyes, acidifiers, antioxidants and the like, with the proviso that the constituents have at least adequate solubility in water. Accordingly, individual constituents may belong both to the water-insoluble phase and to the water-soluble phase, depending on the water solubility of the special representatives. However, combinations may also be used, for example a combination of a water-soluble and a water-insoluble emulsifier, in which case the individual representatives are present in different phases. The water-insoluble component usually makes up 5 to 95% by weight and preferably 20 to 80% by weight of the preparation.

Water-soluble softeners or plasticizers are added to the chewing gum compositions to improve chewability and the chewing feel and are present in the mixtures in quantities of typically 0.5 to 15% by weight. Typical examples are glycerol, lecithin and aqueous solutions of sorbitol, hydrogenated starch hydrolysates or corn sirup.

Fillers are particularly suitable for the production of low-calorie chewing gums and may be selected, for example, from polydextrose, raftilose, raftilin, fructo-oligosaccharides (NutraFlora), palatinose oligosaccharides, guar gum hydrolyzates (Sun Fiber) and dextrins.

The chewing gums may additionally contain auxiliaries and additives which are suitable, for example, for dental care, more particularly for controlling plaque and gingivitis, such as for example chlorhexidine, CPC or triclosan. They may also contain pH adjusters (for example buffer or urea), anti-caries agents (for example phosphates or fluorides), biogenic agents (antibodies, enzymes, caffeine, plant extracts), providing these substances are permitted in foods and do not undesirably interact with one another.

### Candies

According to the present invention the preferred candies are so-called hard-boiled candies. Their bases are usually prepared from a mixture of sugar and other carbohydrates that are kept in an amorphous or glassy condition. This form can be considered a solid syrup of sugars generally having up to about 4.5 wt.-percent moisture, based on the weight of the candy base, with about 0.5 to about 2.5 wt.-percent being preferred and about 1.0 to about 1.5 wt.-percent being most preferred. Such materials normally contain up to 65 wt.-percent corn syrup, up to 80 wt.-percent sugar and from 0.1 to 5.0 wt.-percent water. Generally, the ratio of sugar (or other sweetener suitable for candy formulation) to corn syrup is within the range of about 70:25 to about 45:55 with about 60:40 being preferred. The syrup component generally is prepared from corn syrups high in fructose but may include other materials. Further ingredients such as flavorings, sweeteners, acidulants, colorants and so forth may also be added.

Hard boiled candy bases may also be prepared from non-fermentable sugars such as sorbitol, mannitol, xylitol, maltitol, hydrogenated starch hydrolysate, hydrogenated corn syrup and mixtures thereof. The candy bases may contain up to about 95% sorbitol, a mixture of sorbitol and mannitol at a ratio of about 9.5 to 0.5 up to about 7.5 to 2.5 and hydrogenated corn syrup up to about 55% of the syrup component.

### Compressed Tablets

According to the present invention the oral compositions can represent compressed tablets, comprising the liquid flavor in amounts of typically about 0.1 to about 0.6 wt.-percent and preferably about 0.5 wt.-percent

### Additives

The oral compositions of the present invention may include additional additives as for examples aroma compounds, sweeteners or vitamins, in amounts of from about 0.1 to about 10 wt.-percent These additives may also represent components of the respective medicaments.

### Flavor Compounds

Aroma compounds are selected from Anethol, Acetanisol, Acetaldehyde, Acetylmethylcarbinol, 2-Acetylpyrazine, 2-Acetylpyridine, 2-Acethylthiazoline, 2-Acethylthiazole, Allylcapronate, alpha-Amylcinnamicaldehyde, para-Anisaldehyde, Anisalcohol, Dimethylisopropyldithiazine, Benzaldehyde, Benzylacetate, l-Borneol, Butiric acid, Butylacetate, 3-Butylidenphtalide, Capric acid, l-Carvone, d-Carvone, Carvomenthone, cis-Carvylacetate, Caryophyllene, 1,8-Cineol, 1,4-Cineol, Cinnamylacetate, Citral, Citronellal, Citronellol, Citronellylacetate, Cuminaldehyde, Cyclopentadecanolide, alpha-Damascone, beta-Damascone, alpha-Damascenone, beta-Damascenone, delta-Decalactone, gamma-Decalactone, Dehydromenthofurolactone, Dihydromenthofurolactone, 2,3-Diethylpyrazine, Dihydroanethol, Dihydrocarvone, Dihydrocoumarine, beta-Dihydroionone, Dimethylanthranilate, Dimethylsulfide, Dimethylpyrazine, Sotolon, Diphenyloxide, Divanilline, 2,4-Decadienal, delta-Dodecalacton, gamma-Dodecalactone, Acetic acid, Ethylacetate, Ethylbutyrate, 2-Ethylmethylbutyrate, Ethylcapronate, Ethylcaprylate, Ethylcinnamate, Ethylisobutyrate, Ethylvanilline, Ethyllactate, Ethylmaltol, Ethylmethylthiopropionate, 4-Ethylphenol, Ethylisovalerianate, Eugenol, Fenchol, Furaneol, Filbertone, Frambinone, Frambinonmethylether, Furfurylthiol, Undecatriene, Geraniol, Geranylacetate, Geranylisobutyrate, Guaiacol, Heliotropine, 2-Heptanone, 4-Z-Heptenal, gamma-Hexalactone, gamma-Heptalactone, 3-Z-Hexenol, 2-E-Hexenol, Hexanol, Hexylacetate, 3-Z-Hexenylacetate, 2-E-Hexenylacetate, alpha-Hexylcinnamicaldehyde, 3-ZHexenylcapronate, Hotrienol, Indol, alpha-Irone, alpha-Ionone, beta-Ionone, Isoamylacetate, 20) Isoamylbutyrate, Isoamylisovaerianate, Isobutylacetate, Isobutylthiazol, Isobutyraldehyde, Isovaleraldehyde, Isoeugenol, Isomenthone, Isopropylmethoxypyrazine, Isobutylmethoxypyrazine, 2,4-Isopropylmethylpyrazine, Isopulegol, Jasminlactone, cis-Jasmone, Campher, Ketoisophorone, Cresol, d-Limonene, l-Linalool, d-Linalool, Linalylacetate, Linalooloxide, Maltol, Methylcyclopentenolone, L-Menthone, D-Menthone, L-Menthol, D-Menthol, Neo Menthol, L-25 Menthylacetate, D-Menthylacetate, Massoilactone, Melonal, 1,8-Menthenthiol, 1,8-Epithiomenthane, 8,3-Thiomenthanone, Menthofurolactone, Menthadienylacetate, 2,3-Methoxymethylpyrazine, Methylanthranilate, Methylsalicylate, Thymol, Methylbutyrate, 2-Methylbutylacetate, Methylcinnamate, 2,3-Methylfuranthiol, 2,3-Methyltetrahydrofuranthiol, Methyljasmonate, Methyldihydrojasmonate, Methylthiobutyrate, 1,3-Methylthiohexylacetate, 1,3-Methylthiohexanol, Methional, Myrtenal, Naringin, Neral, Nerol, Nerylacetate, gammaNonalactone, delta-Nonalactone, 2-E-Nonenal, 6-Z-Nonenal, 6-Z-Nonenol, Nootkatone, Dihydronootkatone, 1,3-Octenol, gamma-Octalactone, delta-Octalactone, Pelitorine, 1,3-Pentenone, Pentylacetate, Phenylacetaldehyde, Phenylethylalcohol, Phenylethylacetate, Piperitanat, Prenylthiol, Prenylthioacetate, Rosenoxide, Rubenamine, Rubescenamine, Sabinenhydrate, Skatole, Styrolylacetate, Terpineol, 4-Terpinenol, 1,3-Thiohexanol, 1,3-Thiohexylacetate, 2,4,4-Thiopentanone, Trimethylpyrazine, delta-Undecalactone, gammaUndecalactone, 2,4-Decadienal, 2,4-Nonadienal, 2,6-Nonadienal, 2,4-Undecadienal, Vanilline, Vinylguaiacol, Whiskylactone, Cinnamicaldehyde, Cinnamicalcohol, Diallyldisulfide, Allylisothiocyanate, Hexanal, E-2-Hexenal, Octanal, Decanal, Tridecatrienal, 12-Methyltridecanal, alpha-Pinene, beta-Pinene, Piperitone, as well as their mixtures.

### Sweeteners

Suitable sweet-tasting substances, including natural sources of these substances (component e5), such as for example sweet-tasting carbohydrates or sugars (e.g. sucrose (synonymous with saccharose), trehalose, lactose, maltose, melezitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin) or vegetable preparations containing predominantly these carbohydrates (e.g. from sugar beet (Beta vulgaris ssp., sugar fractions, sugar syrup, molasses), from sugar cane (Saccharum officinarum ssp., e.g. molasses, sugar syrups), from sugar maple (Acer ssp.), from agave (agave thick juice), synthetic/enzymatic hydrolysates of starch or sucrose (e.g. invert sugar syrup, highly enriched fructose syrups made from corn starch), fruit concentrates (e.g. from apples or pears, apple syrup, pear syrup), sugar alcohols (e.g. erythritol, threitol, arabitol, ribitol, xylitol, sorbitol, mannitol, dulcitol, lactitol), proteins (e.g. miraculin, monellin, thaumatin, curculin, brazzein), sweeteners (magap, sodiumcyclamate, acesulfame K, neohesperidin dihydrochalcone, saccharin sodium salt, Aspartame®, superaspartame, neotame, alitame, sucralose, stevioside, rebaudioside, lugduname, carrelame, sucrononate, sucrooctate, monatin, phyllodulcin), certain sweet-tasting amino acids (glycine, D-leucine, D-threonine, D-asparagine, Dphenylalanine, D-tryptophan, L-proline), other sweet-tasting low-molecular substances (e.g. hernandulcin, dihydrochalcone glycosides, glycyrrhizin, glycyrrhetinic acid ammonium salt or other glycyrrhetinic acid derivatives), liquorice extracts (Glycyrrhizza glabra ssp.), Lippia dulcis extracts, Momordica ssp. extracts or individual substances (in particular Momordica grosvenori [Luo Han Guo] and the mogrosides obtained therefrom), Hydrangea dulcis or Stevia ssp. (e.g. Stevia rebaudiana) extracts or individual substances.

### Vitamins

In another embodiment of the present invention the compositions may include vitamins. Vitamins have diverse biochemical functions. Some have hormone-like functions as regulators of mineral metabolism (e.g., vitamin D), or regulators of cell and tissue growth and differentiation (e.g., some forms of vitamin A). Others function as antioxidants (e.g., vitamin E and sometimes vitamin C). The largest numbers of vitamins (e.g. B complex vitamins) act as precursors for enzyme cofactors that help enzymes in their work as catalysts in metabolism. In this role, vitamins may be tightly bound to enzymes as part of prosthetic groups: For example, biotin is part of enzymes involved in making fatty acids. Vitamins may also be less tightly bound to enzyme catalysts as coenzymes, detachable molecules that function to carry chemical groups or electrons between molecules. For example, folic acid carries various forms of carbon group—methyl, formyl, and methylene—in the cell. Although these roles in assisting enzyme-substrate reactions are vitamins' best-known function, the other vitamin functions are equally important. In the course of the present invention suitable vitamins are selected from the group consisting of.
Vitamin A (retinol, retinal, beta carotene),
Vitamin B₁ (thiamine),
Vitamin B₂ (riboflavin),
Vitamin B₃ (niacin, niacinamide),
Vitamin B₅ (panthothenic acid),
Vitamin B₆ (pyridoxine, pyridoxamine, paridoxal),
Vitamin B₇ (biotin),
Vitamin B₉ (folic acid, folinic acid),
Vitamin B₁₂ (cyanobalamin, hydoxycobalmin, methylcobalmin),
Vitamin C (ascorbic acid),
Vitamin D (cholecalciferol),
Vitamin E (tocopherols, tocotrienols), and
Vitamin K (phyolloquinone, menaquinone).

The preferred vitamins are ascorbic acid and tocopherols. Said vitamins may be present in the food composition in amounts of about 0.1 to about 5 wt.-percent, and preferably about 0.5 to about 1 wt.-percent

### INDUSTRIAL APPLICATION

Another object of the present invention refers to a method for fighting microorganisms causing biofilms and/or plaque in the oral cavity comprising or consisting of the following steps:
(a) providing a mixture as described above;
(b) incorporating said mixture into an oral composition; and
(c) bringing said oral composition in contact with the oral cavity of a human.

The invention also encompasses the use of the mixture as described above for oral care applications, namely fighting of microorganisms causing biofilms and/or plaque in the oral cavity. In the context of the present invention oral care applications refers to products such as toothpastes, mouthwashes, (dental) chewing gums and candies.

In a preferred embodiment the mixture contains or consists of carvacrol as the flavoring agent and menthyl lactate, menthyl propylene glycol carbonate, 3-(−) menthoxypropane-12-diol or mixtures thereof.

For the sake of good order, it is emphasized that all preferred embodiments, ranges, combinations and the like mentioned above refer as well to the claimed method and uses. A repetition is therefore not necessary.

### EXAMPLES

### Examples 1 to 23 (Table 1-3)

### Determination of Minimum Inhibitory Concentration (MIC) Data In Vitro

MIC data were evaluated based on DIN 58940-8 micro dilution method. For the experiments three relevant bacteria occurring in the oral biofilm were selected: Streptococcus mutans (DSM 20523, ATCC 25175), Porphyromonas gingivalis (DSM 20709, ATCC33277), and Prevotella intermedia (DSM 20706, ATCC25611).

All test organisms were cultivated in suitable complex medium and under anaerobic and/or microaerophilic conditions. Every test substance was applied as technical triplicate on a 96-microtiter plate in a total volume of 300 μL per well. The inoculum was directly transferred from a glycerin stock solution or from a freshly prepared preculture. Therefore 15 mL reagent tubes were filled with specific complex growth medium and inoculated with skim milk stocks of single strains. The incubation was done at 37° C. up to 48 h until an OD₆₂₀>0.1-1.0. Suspensions were controlled microscopically. In both cases the bacteria were transferred and diluted to fresh complex medium on a 96 well plate.

Test substances were either the pure materials or mixtures. The mixtures contain a cooling compound and an aroma component according to claims. Further these effective building blocks can be included in an aroma composition. Therefore, nature identical (synthetic) bases (base nature identical A/B) and/or natural basic formulations (base natural A/B) can be added.

The mixtures contained either Carvacrol (synthetic material) or the natural oregano oil with a typical Carvacrol content in the range of 50-80%.

Test substances were 100-fold solved in dimethyl sulfoxide (DMSO) and diluted directly on the 96 well plate. As negative controls farnesol and/or triclosan were applied in appropriate concentrations from 1 to 100 ppm. An untreated medium control was applied as growth control. The incubation of the test plates was done for 16-24 h at 37° C. At the end of the experiment, growth was determined photometrically by detecting OD₆₂₀ values using Sunrise Photometer (TECAN, Austria). Mean values of triplicates were calculated and normalized with blank values (growth medium with or without test substances). The growths of treated wells were evaluated in relation to the growth control. Minimum inhibition concentrations (MIC) were defined as minimal concentration (in ppm) that causes a complete inhibition of bacterial growth.

Synergies were calculated using the following formula. The formula can be modified according to the number of components per mixture. MIC stands for minimum inhibitory concentration with the dimension of parts per million [ppm].

$$Synergistic Index ( SI ) = \frac{MICmix * PA}{MIC A} + \frac{M ICmix * PB}{MIC B}$$

MICmix=MIC value of mixture [ppm]
PA=Percentage of component A [%]
MIC A=MIC value of component A [ppm]
PB=Percentage of component B [%]
MIC B=MIC value of component B [ppm]
   A synergism is given if the resulting value is below 1.

### Ex Vivo Biofilm Model

The experiment was developed following the method described in WO2018/033211 and Kistler et al (Kistler et al. 2015).

Up to 50 subjects were recruited for saliva donation. Participants were between 18 and 65 years old and medically healthy. Subjects who had taken antibiotics, ate antimicrobial food such as garlic within last 24 h and/or had suffer from any systemic conditions that may affected their immune or inflammatory status, were excluded from the study. Fresh morning saliva from all donors were selected and pooled. All volunteers were asked to refrain from drinking, eating, or brushing their teeth bevor donating by expectoration into sterile universal tubes.

The Calgary Biofilm Device (CBD) was originally developed for medical usage. It is commercially available as MBEC (Minimum Biofilm Eradication Concentration) Biofilm Inoculator and it is suitable as a basis for biofilm research (Kistler et al. 2015; Ceri et al. 1999). The 96 pegs of the device lid were coated with hydroxylapatite and mimic teeth. It was fitted to a second 96 well plate in a way that the pegs were in contact with saliva. The CBD was incubated under anaerobic conditions for 18 hours at 37° C. After that the lid was transferred to a new baseplate containing Oral care medium (Brain Heart Infusion broth (Merck) supplemented with pig stomach mucin (1 g/L), haemin (10 mg/L), and menadion (0.5 mg/L). The growth medium was changed every day.

Stock solutions of all test substances were prepared in dimethylsulfoxide (DMSO). For the working concentration the stocks were diluted in NaCl-solution or other solvents. If necessary, a treatment in the sonification bath or heater could be applied. Pure NaCl-solution was used as positive biofilm growth control. The treatment was done 4 times within 2 days for 2 min at 37° C. and 300 rpm. After the treatment the pegs were washed with pure NaCl solution under same conditions and then again transferred to the growth medium. Six replicates were applied on each plate for every test substance and concentration. At the end the pegs were washed again, and microbial activity was quantified photometrically by fluorescein diacetate (FDA) hydrolysis. FDA is converted into fluorescein by protease, lipase, and esterase activities in the biofilm, thus the quantification of fluorescein represents the activity of the biofilm.

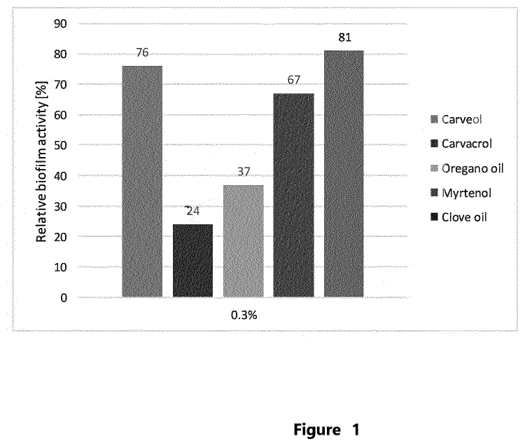

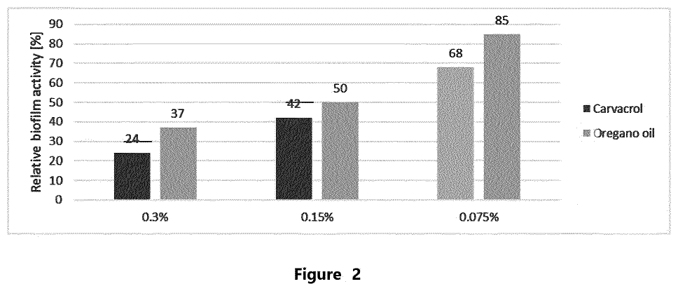

Table 1 to 3 provides the results for minimal inhibition concentrations for various mixtures of carvacrol or oregano oil and physiological cooling agents and the related synergy indices. FIG. 1 shows results for the relative biofilm activity after respective treatments of several single components.

### REFERENCES

Kistler, James O.; Pesaro, Manuel; Wade, William G. (2015): Development and pyrosequencing analysis of an in-vitro oral biofilm model. BMC microbiology 15, p. 24.
Ceri, H.; Olson, M. E.; Stremick, C.; Read, R. R.; Morck, D.; Buret, A. (1999): The Calgary Biofilm Device: new technology for rapid determination of antibiotic susceptibilities of bacterial biofilms. Journal of clinical microbiology 37 (6), p. 1771-1776.

TABLE 1
Antimicrobial efficacy and synergisms for various combinations, calculated for different
bacteria strains, calculated with measured MIC value [ppm] of the mixture (MIC
mix, [ppm]), measured MIC value of component A (MIC A, [ppm]), percentage
of component A in the mixture (PA), measured MIC value of component B (MIC B, [ppm]),
percentage of component B in the mixture (PB), calculated synergistic indices (SI)

| | Combination of carvacrol | | MIC | MIC | | MIC | | | |
|---|---|---|---|---|---|---|---|---|---|
| Ex | and secondary agent | Ratio | mix | A | PA | B | PB | SI | Strain |
| 1 | 50% Carvacrol + 50% | 1:1 | 125 | 250 | 0.5 | 125 | 0.5 | 0.75 | P. gingivalis |
| | Menthyl lactate | | | | | | | | |
| 2 | 20% Carvacrol + 80% | 20:80 | 64 | 250 | 0.2 | 125 | 0.8 | 0.46 | P. gingivalis |
| | Menthyl lactate | | | | | | | | |
| 3 | 80% Carvacrol + 20% | 80:20 | 125 | 250 | 0.8 | 125 | 0.2 | 0.60 | P. gingivalis |
| | Menthyl lactate | | | | | | | | |
| 4 | 50% Carvacrol + 50% | 1:1 | 125 | 250 | 0.5 | 125 | 0.5 | 0.75 | P. intermedia |
| | Menthyl lactate | | | | | | | | |
| 5 | 20% Carvacrol + 80% | 20:80 | 125 | 250 | 0.2 | 125 | 0.8 | 0.90 | P. intermedia |
| | Menthyl lactate | | | | | | | | |
| 6 | 80% Carvacrol + 20% | 80:20 | 125 | 250 | 0.8 | 125 | 0.2 | 0.60 | P. intermedia |
| | Menthyl lactate | | | | | | | | |
| 7 | 50% Carvacrol + 50% (−)- | 1:1 | 64 | 250 | 0.5 | 125 | 0.5 | 0.38 | P. gingivalis |
| | Menthyl propylene glycol | | | | | | | | |
| | carbonate | | | | | | | | |
| 8 | 20% Carvacrol + 80% (−)- | 20:80 | 64 | 250 | 0.2 | 125 | 0.8 | 0.46 | P. gingivalis |
| | Menthyl propylene glycol | | | | | | | | |
| | carbonate | | | | | | | | |
| 9 | 80% Carvacrol + 20% (−)- | 80:20 | 125 | 250 | 0.8 | 125 | 0.2 | 0.60 | P. gingivalis |
| | Menthyl propylene glycol | | | | | | | | |
| | carbonate | | | | | | | | |
| 10 | 50% Carvacrol + 50% 3- | 1:1 | 250 | 250 | 0.5 | 500 | 0.5 | 0.75 | S. mutans |
| | (−)-Menthoxy)propane- | | | | | | | | |
| | 1,2-diol | | | | | | | | |
| 11 | 50% Carvacrol + 50% | 1:1 | 125 | 250 | 0.5 | 125 | 0.5 | 0.75 | S. mutans |
| | Menthyl propylene glycol | | | | | | | | |
| | carbonate | | | | | | | | |
| 12 | 80% Carvacrol + 20% | 80:20 | 250 | 250 | 0.8 | 500 | 0.2 | 0.9 | S. mutans |
| | D,L-Menthone glyceryl | | | | | | | | |
| | ketal | | | | | | | | |
| 13 | 80% Carvacrol + 20% | 80:20 | 125 | 250 | 0.8 | 250 | 0.2 | 0.5 | P. gingivalis |
| | D,L-Menthone glyceryl | | | | | | | | |
| | ketal | | | | | | | | |
| 14 | 80% Carvacrol + 20% | 80:20 | 125 | 250 | 0.8 | 250 | 0.2 | 0.5 | P. intermedia |
| | D,L-Menthone glyceryl | | | | | | | | |
| | ketal | | | | | | | | |

TABLE 2
Antimicrobial efficacy and synergisms for specific synthetic mixtures (product examples), calculated
with measured MIC value [ppm] of the mixture (MIC mix, [ppm]), measured MIC value of component
A (MIC A, [ppm]), percentage of component A in the mixture (PA), measured MIC value of component
B (MIC B, [ppm]), percentage of component B in the mixture (PB), measured MIC value of component
C (MIC C, [ppm]), percentage of component C in the mixture (PC), calculated synergistic indices (SI)

| | Combination of carvacrol | | MIC | MIC | | MIC | | MIC | | | |
|---|---|---|---|---|---|---|---|---|---|---|---|
| Ex | and secondary agent | Ratio | mix | A | PA | B | PB | C | PC | SI | Strain |
| 15 | 25% Carvacrol, | ′25:25:50 | 250 | 250 | 0.25 | 250 | 0.25 | 500 | 0.5 | 0.75 | S. mutans |
| | 25% Menthyl lactate, | | | | | | | | | | |
| | 50% base nature identical | | | | | | | | | | |
| | (synthetic) A | | | | | | | | | | |
| 16 | 25% Carvacrol, | ′25:25:50 | 125 | 250 | 0.25 | 125 | 0.25 | 125 | 0.5 | 0.88 | P. gingivalis |
| | 25% Menthyl lactate, | | | | | | | | | | |
| | 50% base nature identical | | | | | | | | | | |
| | (synthetic) A | | | | | | | | | | |
| 17 | 25% Carvacrol, | ′25:25:50 | 250 | 250 | 0.25 | 250 | 0.25 | 500 | 0.5 | 0.75 | S. mutans |
| | 25% Menthyl lactate, | | | | | | | | | | |
| | 50% base nature identical | | | | | | | | | | |
| | (synthetic) B | | | | | | | | | | |
| 18 | 25% Carvacrol, | ′25:25:50 | 125 | 250 | 0.25 | 125 | 0.25 | 125 | 0.5 | 0.88 | P. gingivalis |
| | 25% Menthyl lactate, | | | | | | | | | | |
| | 50% base nature identical | | | | | | | | | | |
| | (synthetic) B | | | | | | | | | | |

TABLE 3
Antimicrobial efficacy and synergisms for specific natural mixtures (product examples), calculated
with measured MIC value of the mixture (MIC mix), measured MIC value of component A (MIC
A), percentage of component A in the mixture (PA), measured MIC value of component B (MIC
B), percentage of component B in the mixture (PB), measured MIC value of component C (MIC
C), percentage of component C in the mixture (PC), calculated synergistic indices (SI).

| | Combination of carvacrol | | MIC | MIC | | MIC | | MIC | | | |
|---|---|---|---|---|---|---|---|---|---|---|---|
| Ex | and secondary agent | Ratio | mix | A | PA | B | PB | C | PC | SI | Strain |
| 19 | 36% Oregano oil, | ′36:25:39 | 250 | 250 | 0.36 | 250 | 0.25 | 500 | 0.39 | 0.81 | S. mutans |
| | 25% Menthyl lactate (NAT.), | | | | | | | | | | |
| | 39% base natural A | | | | | | | | | | |
| 20 | 36% Oregano oil, | ′36:25:39 | 64 | 125 | 0.36 | 125 | 0.25 | 125 | 0.39 | 0.51 | P. gingivalis |
| | 25% Menthyl lactate (NAT.), | | | | | | | | | | |
| | 39% base natural A | | | | | | | | | | |
| 21 | 36% Oregano oil, | ′36:25:39 | 250 | 250 | 0.362 | 250 | 0.25 | 500 | 0.388 | 0.81 | S. mutans |
| | 25% Menthyl lactate (NAT.), | | | | | | | | | | |
| | 39% base natural B | | | | | | | | | | |
| 22 | 36% Oregano oil, | ′36:25:39 | 64 | 125 | 0.362 | 125 | 0.25 | 125 | 0.388 | 0.51 | P. gingivalis |
| | 25% Menthyl lactate (NAT.), | | | | | | | | | | |
| | 39% base natural B | | | | | | | | | | |

FIG. 1 shows effective oral biofilm reduction. The FIGURE shows the results for the relative biofilm activity after respective treatments with various flavouring agents. 100% represent the activity of the untreated control biofilm.

FIG. 1 shows concentration dependent oral biofilm reduction after respective treatment with Carvacrol and Oregano oil. The FIGURE shows the remaining relative biofilm activity compared to control after the treatments.

### FORMULATION EXAMPLES

The following Tables 4 to 17 provide various examples for oral compositions

TABLE 4
Chewing gum, free of sugar; all amounts in wt.-percent

| Composition | A | B | C |
|---|---|---|---|
| Gum base | 30.00 | 30.00 | 30.00 |
| Sorbit, powdered | To 100 | To 100 | To 100 |
| Isomalt, powdered | 9.50 | 9.50 | 9.50 |
| Xylitol | 2.00 | 2.00 | 2.00 |
| Mannit D | 3.00 | 3.00 | 3.00 |
| Aspartame | 0.10 | 0.10 | 0.10 |
| Acesulfam K | 0.10 | 0.10 | 0.10 |
| Emulgum / Plasticizing agent | 0.30 | 0.30 | 0.30 |
| Sorbitol (70% water) | 13.00 | 13.00 | 13.00 |
| Glycerol | 1.00 | 1.00 | 1.00 |
| Flavor | 0.998 | 0.98 | 0.8 |
| Mixture Example 1 | 0.002 | | |
| Mixture Example 1 | | 0.02 | |
| Mixture Example 1 | | | 0.2 |

| | |
|---|---|
| Total | 100 |

TABLE 5
Tooth paste; all amounts in wt.-percent

| Composition | A | B | C |
|---|---|---|---|
| Deionised water | To 100 | To 100 | To 100 |
| Glycerol | 20.00 | 20.00 | 20.00 |
| Solbrol M (sodium salt) | 0.15 | 0.15 | 0.15 |
| Sodium monofluor phosphate | 0.76 | 0.76 | 0.76 |
| Saccharin | 0.20 | 0.20 | 0.20 |
| Dicalciumphosphate dihydrate | 36.00 | 36.00 | 36.00 |
| Aerosil 200 | 3.00 | 3.00 | 3.00 |
| Sodium carboxymethyl cellulose | 1.20 | 1.20 | 1.20 |
| Sodium lauryl sulfate | 1.30 | 1.30 | 1.30 |
| Flavor | 0.997 | 0.97 | 0.7 |
| Mixture Example 7 | 0.003 | | |
| Mixture Example 4 | | 0.03 | |
| Mixture Example 19 | | | 0.3 |

| | |
|---|---|
| Total | 100 |

TABLE 6
Dentifrice with therapeutic dose of
fluoride; all amounts in wt.-percent

| | SnF₂ | NaF | NaMFP |
|---|---|---|---|
| Ingredients | toothpaste | toothpaste | toothpaste |
| Sorbitol (70%) | 46.00 | 65.51 | — |
| USP water | 26.66 | 13.78 | 60.01 |
| Sodium Fluoride | — | 0.24 | — |
| Stannous Fluoride | 0.45 | — | — |
| Sodium Monofluoro- | — | — | 0.76 |
| phosphate | | | |
| Stannous Chloride | 0.56 | — | — |
| Sodium citrate | 1.55 | — | — |
| Sodium Gluconate | 1.3 | — | — |
| Trisodium phosphate | — | 1.10 | 0.42 |
| Monosodium phosphate | — | 0.42 | 0.08 |
| Tetrabasic Sodium | — | — | 0.60 |
| Pyrophosphate | — | | |
| Hydrated Silica | 17.50 | 15.00 | 2.62 |
| Calcium Carbonate | — | — | 32.00 |
| Carbomer | — | 0.30 | — |
| Cellulose Gum | — | 0.75 | 0.92 |
| Carrageenan | 1.05 | — | 1.20 |
| Xanthan Gum, NF | 0.61 | — | — |
| Sodium Saccharin | 0.25 | 0.25 | 0.25 |
| Sodium lauryl sulfate | 1.29 | 1.29 | 1.29 |
| Sodium Hydroxide | 0.75 | — | — |
| (50%) | | | |
| Titanium Dioxide | — | 0.55 | — |
| Flavor | 1.17 | 0.76 | 0.80 |
| Mixture Example 15 | 0.03 | | |
| Mixture Example 15 | | 0.04 | |

TABLE 7
Dentifrice therapeutic dose of fluoride; all amounts in wt.-percent

| | SnF₂ | NaF | NaMFP |
|---|---|---|---|
| Ingredients | toothpaste | toothpaste | toothpaste |
| Sorbitol (70%) | 46.00 | 65.51 | — |
| USP water | 26.66 | 13.78 | 60.01 |
| Sodium Fluoride | — | 0.24 | — |
| Stannous Fluoride | 0.45 | — | — |
| Sodium Monofluoro- | — | — | 0.76 |
| phosphate | | | |
| Stannous Chloride | 0.56 | — | — |
| Sodium citrate | 1.55 | — | — |
| Sodium Gluconate | 1.3 | — | — |
| Trisodium phosphate | — | 1.10 | 0.42 |
| Monosodium phos- | — | 0.42 | 0.08 |
| phate | | | |
| Tetrabasic Sodium | — | — | 0.60 |
| Pyrophosphate | — | | |
| Hydrated Silica | 17.50 | 15.00 | 2.62 |
| Calcium Carbonate | — | — | 32.00 |
| Carbomer | — | 0.30 | — |
| Cellulose Gum | — | 0.75 | 0.92 |
| Carrageenan | 1.05 | — | 1.20 |
| Xanthan Gum, NF | 0.61 | — | — |
| Sodium Saccharin | 0.25 | 0.25 | 0.25 |
| Sodium lauryl sulfate | 1.29 | 1.29 | 1.29 |
| Sodium Hydrxide | 0.75 | — | — |
| (50%) | | | |
| Titanium Dioxide | — | 0.55 | — |
| Flavor | 1.17 | 0.76 | 0.80 |
| Mixture Example 17 | 0.03 | | |
| Mixture Example 17 | | 0.04 | |
| Mixture Example 17 | | | 0.05 |

TABLE 8
Dentifrice therapeutic dose of fluoride; all amounts in wt.-percent

| | SnF₂ | NaF | NaMFP |
|---|---|---|---|
| Ingredients | toothpaste | toothpaste | toothpaste |
| Sorbitol (70%) | 46.00 | 65.51 | — |
| USP water | 26.66 | 13.78 | 60.01 |
| Sodium Fluoride | — | 0.24 | — |
| Stannous Fluoride | 0.45 | — | — |
| Sodium Monofluoro- | — | — | 0.76 |
| phosphate | | | |
| Stannous Chloride | 0.56 | — | — |
| Sodium citrate | 1.55 | — | — |
| Sodium Gluconate | 1.3 | — | — |
| Trisodium phosphate | — | 1.10 | 0.42 |
| Monosodium phosphate | — | 0.42 | 0.08 |
| Tetrabasic Sodium | — | — | 0.60 |
| Pyrophosphate | — | | |
| Hydrated Silica | 17.50 | 15.00 | 2.62 |
| Calcium Carbonate | — | — | 32.00 |
| Carbomer | — | 0.30 | — |
| Cellulose Gum | — | 0.75 | 0.92 |
| Carrageenan | 1.05 | — | 1.20 |
| Xanthan Gum, NF | 0.61 | — | — |
| Sodium Saccharin | 0.25 | 0.25 | 0.25 |
| Sodium lauryl sulfate | 1.29 | 1.29 | 1.29 |
| Sodium Hydrxide (50%) | 0.75 | — | — |
| Titanium Dioxide | — | 0.55 | — |
| Flavor | 1.17 | 0.76 | 0.80 |
| Mixture Example 19 | 0.03 | | |
| Mixture Example 19 | | 0.04 | |
| Mixture Example 19 | | | 0.05 |

TABLE 9
Toothpaste with subtherapeutic dose of fluoride and subtherapeutic dose
of a second anti plaque agent blend, all amounts in wt.-percent

| | MFP/ | | SnF₂ | | NaF/ | |
|---|---|---|---|---|---|---|
| | nature | MFP/ | nature | SnF₂ | nature | NaF/ |
| Ingredients | identical | natural | identical | natural | identical | natural |
| Sorbitol (70%) | 36.98 | 36.98 | 46.12 | 46.12 | 65.75 | 65.75 |
| USP water | 13 | 13 | 26.80 | 26.80 | 13.50 | 13.50 |
| Sodium Fluoride | — | — | — | — | 0.06 | 0.06 |
| Stannous Fluoride | — | — | 0.11 | 0.11 | — | — |
| Sodium Mono- | 0.19 | 0.19 | — | — | — | — |
| fluorosphosphate | | | | | | |
| Stannous Chloride | 1.10 | 1.10 | 0.56 | 0.56 | — | — |
| Sodium citrate | — | — | 1.55 | 1.55 | — | — |
| Sodium Gluconate | 1 | 1 | 1.30 | 1.30 | — | — |
| Trisodium phosphate | — | — | — | — | 1.10 | 1.10 |
| Monosodium phosphate | — | — | — | — | 0.42 | 0.42 |
| Tetrabasic | — | — | — | — | — | — |
| Sodium Pyrophosphate | — | — | 17.50 | 17.50 | 15.00 | 15.00 |
| Hydrated Silica | | | | | | |
| Calcium Carbonate | 32 | 32 | — | — | — | — |
| Carbomer | — | — | — | — | 0.30 | 0.30 |
| Cellulose Gum | 1 | 1 | — | — | 0.75 | 0.75 |
| Carrageenan | — | — | 1.05 | 1.05 | — | — |
| Xanthan Gum, NF | 0.30 | 0.30 | 0.61 | 0.61 | — | — |
| Sodium Saccharin | 0.5 | 0.5 | 0.45 | 0.45 | 0.13 | 0.13 |
| Sodium lauryl sulfate | 1.29 | 1.29 | 1.50 | 1.50 | 1.20 | 1.20 |
| Stevia Glycosides | — | — | — | — | — | — |
| Sodium Hydroxide (50%) | 0.33 | 0.33 | 0.75 | 0.75 | — | — |
| Titanium Dioxide | — | — | — | — | 0.55 | 0.55 |
| Sodium bicarbonate | 10 | 10 | — | — | — | — |
| Flavor | 1.45 | 1.45 | 1.65 | 1.65 | 1.25 | 1.25 |
| Mixture Example 16 | 0.1 | | 0.03 | | 0.05 | |
| Mixture Example 18 | | 0.1 | | 0.03 | | 0.05 |

TABLE 10
Exemplary formulation for a tooth paste; all amounts in wt.-percent

| | TP1 | TP2 | TP3 | TP4 |
|---|---|---|---|---|
| 70% Sorbitol | 25 | 25 | 25 | 25 |
| Silica | 10 | 5 | 3 | 3 |
| Hydroxyethylcellulose | 0.5 | 0.5 | 0.5 | 0.5 |
| Hydroxyethylcellulose | | | | |
| Xanthan Gum | 0.5 | 0.5 | 0.5 | 0.5 |
| Sodium Lauryl Slfate | 0.2 | 0.2 | 0.2 | 0.2 |
| Titanium Oxide | 0.2 | 0.2 | 0.2 | 0.2 |
| Sodium Saccharine | 0.1 | 0.1 | 0.1 | 0.1 |
| Methyl p- | 1 | 1 | 1 | 1 |
| hydroxybenzoate | | | | |
| Sodium Bicarbonate | 40 | 45 | 50 | 55 |
| Mixture Example 1 | 0.01 or 0.02 or | 0.01 or 0.02 or | 0.01 or 0.02 or | 0.01 or 0.02 or |
| | 0.03 or 0.04 or | 0.03 or 0.04 or | 0.03 or 0.04 or | 0.03 or 0.04 or |
| | 0.05 | 0.05 | 0.05 | 0.05 |
| Water | ad 100 | ad 100 | ad 100 | ad 100 |

TABLE 11
Exemplary formulation for a tooth paste; all amounts in wt.-percent

| | TP5 | TP6 | TP7 | TP8 | TP9 |
|---|---|---|---|---|---|
| 70% Sorbitol | 10 | 10 | 10 | 10 | 10 |
| Silica | 10 | 7 | 7 | 5 | 3 |
| Crystalline cellulose | 15 | 15 | 15 | 15 | 15 |
| Lauryl Glucoside | 0.5 | 0.5 | 0.5 | 0.5 | 0.5 |
| Titanium Oxide | 0.2 | 0.2 | 0.2 | 0.2 | 0.2 |
| Sodium Saccharine | 0.1 | 0.1 | 0.1 | 0.1 | 0.1 |
| Methyl p- | 1 | 1 | 1 | 1 | 1 |
| hydroxybenzoate | | | | | |
| Sodium Bicarbonate | 40 | 45 | 50 | 55 | 60 |
| Mixture Example 1 | 0.01or 0.02 or | 0.01or 0.02 or | 0.01or 0.02 or | 0.01or 0.02 or | 0.01or 0.02 or |
| | 0.03 or 0.04 | 0.03 or 0.04 | 0.03 or 0.04 | 0.03 or 0.04 | 0.03 or 0.04 |
| | or 0.05 | or 0.05 | or 0.05 | or 0.05 | or 0.05 |
| Water | ad 100 | ad 100 | ad 100 | ad 100 | ad 100 |

TABLE 12
Exemplary formulation for a tooth paste; all amounts in wt.-percent

| | TP10 | TP11 | TP12 | TP13 | TP14 |
|---|---|---|---|---|---|
| Glycerin | 20 | 20 | 20 | 20 | 20 |
| Calciumcarbonate | 10 | 10 | 10 | 5 | 3 |
| Hydroxyethylcellulose | 0.5 | 0.5 | 0.5 | 0.5 | 0.5 |
| Xanthan Gum | 0.7 | 0.7 | 0.7 | 0.7 | 0.7 |
| Cocoamidopropylbetaine | 0.5 | 0.5 | 0.5 | 0.5 | 0.5 |
| Sodium Saccharine | 0.1 | 0.1 | 0.1 | 0.1 | 0.1 |
| Methyl p- | 1 | 1 | 1 | 1 | 1 |
| hydroxybenzoate | | | | | |
| Sodium Bicarbonate | 40 | 40 | 45 | 55 | 70 |
| Mixture Example 1 | 0.01or 0.02 | 0.01or 0.02 | 0.01or 0.02 | 0.01or 0.02 | 0.01or 0.02 |
| | or 0.03 or | or 0.03 or | or 0.03 or | or 0.03 or | or 0.03 or |
| | 0.04 or 0.05 | 0.04 or 0.05 | 0.04 or 0.05 | 0.04 or 0.05 | 0.04 or 0.05 |
| Water | ad 100 | ad 100 | ad 100 | ad 100 | ad 100 |

TABLE 13
Exemplary formulation for a tooth paste; all amounts in wt.-percent

| | TP15 | TP16 | TP17 | TP18 | TP19 |
|---|---|---|---|---|---|
| Glycerin | 20 | 20 | 20 | 20 | 20 |
| Silica | 30 | 10 | 5 | 5 | 5 |
| Xanthan Gum | 0.5 | 0.5 | 0.5 | 0.5 | 0.5 |
| PEG-60 hydrogenated | 1 | 1 | 1 | 1 | 1 |
| castor iol | | | | | |
| Alkyl Glycoside | 1 | 1 | 1 | 1 | 1 |
| (C8-C16) | | | | | |
| Titanium Oxide | 0.2 | 0.2 | 0.2 | 0.2 | 0.2 |
| Sodium Saccharine | 0.1 | 0.1 | 0.1 | 0.1 | 0.1 |
| Methyl p- | 1 | 1 | 1 | 1 | 1 |
| hydroxybenzoate | | | | | |
| Sodium Bicarbonate | 30 | 45 | 55 | 60 | 60 |
| Mixture Example 1 | 0.01or 0.02 or | 0.01or 0.02 or | 0.01or 0.02 or | 0.01or 0.02 or | 0.01or 0.02 or |
| | 0.03 or 0.04 or | 0.03 or 0.04 or | 0.03 or 0.04 or | 0.03 or 0.04 or | 0.03 or 0.04 or |
| | 0.05 | 0.05 | 0.05 | 0.05 | 0.05 |
| Water | ad 100 | ad 100 | ad 100 | ad 100 | ad 100 |

TABLE 14
Mouth wash concentrate; all amounts in wt.-percent

| Composition | A | B | C |
|---|---|---|---|
| Ethanol 96% | 42.00 | 42.00 | 42.00 |
| Cremophor RH 455 | 5.00 | 5.00 | 5.00 |
| Deionised water | To 100 | To 100 | To 100 |
| Allantoin | 0.20 | 0.20 | 0.20 |
| Sodium saccharin 450 | 0.10 | 0.10 | 0.10 |
| Colour L-Blue 5000 (1% in Wasser) | 0.03 | 0.03 | 0.03 |
| Flavor | 0.13 | 0.19 | 0.20 |
| Mixture Example 3 | 0.002 | | |
| Mixture Example 9 | | 0.002 | |
| Mixture Example 16 | | | 0.002 |

| | |
|---|---|
| Total | 100 |

TABLE 15
Mouth-rinse comprising a therapeutic dose
of fluoride; all amounts in % b.w.

| | Mouth-rinse | Mouth-rinse |
|---|---|---|
| Ingredients | with NaF | with NaF |
| USP water | 99.105 | 99.105 |
| Propylene Glycol | 0.500 | 0.500 |
| Sodium Fluoride | 0.020 | 0.020 |
| Trisodium phosphate decahydrate | — | — |
| Monosodium phosphate monohydrate | — | — |
| Disodium Phosphate heptahydrate | 0.000 | 0.000 |
| Polysorbate 80 | 0.100 | 0.100 |
| Sodium Saccharin | — | — |
| Sodium Hydorxide (50%) | — | — |
| Flavor | 0.100 | 0.100 |
| Benzoic acid | 0.090 | 0.090 |
| Sodium benzoate | 0.020 | 0.020 |
| Phosphoric acid | 0.020 | 0.020 |
| Sucralose | 0.040 | 0.040 |
| Mixture Example 19 | 0.005 | |
| Mixture Example 21 | | 0.005 |

TABLE 16
Hard boiled candy, sugar-free; all amounts in wt.-percent

| Composition | A | B | C |
|---|---|---|---|
| Water | 2.24 | 2.24 | 2.24 |
| Isomalt | To 100 | To 100 | To 100 |
| Xylitol | 2.40 | 2.40 | 2.40 |
| Sucralose | 0.03 | 0.03 | 0.03 |
| Acesulfame K | 0.050 | 0.050 | 0.050 |
| Citric acid | 0.050 | 0.050 | 0.050 |
| Flavor | 0.08 | 0.07 | 0.07 |
| Mixture Example 9 | 0.02 | | |
| Mixture Example 9 | | 0.03 | |
| Mixture Example 6 | | | 0.03 |

| | |
|---|---|
| Total | 100 |

TABLE 17
Hard boiled candy; all amounts in wt.-percent

| Composition | A | B | C |
|---|---|---|---|
| Water | 2.75 | 2.24 | 2.24 |
| Saccharose | To 100 | To 100 | To 100 |
| Glucose syrup | 36.9 | 2.40 | 2.40 |
| Flavor | 0.08 | 0.07 | 0.05 |
| Mixture Example 3 | 0.02 | | |
| Mixture Example 3 | | 0.03 | |
| Mixture Example 15 | | | 0.05 |

| | |
|---|---|
| Total | 100 |

What claimed is:

1. An anti-microbial mixture comprising or (essentially) consisting of:
(a) at least one flavoring agent, and
(b) at least one physiological cooling agent,
wherein said flavoring agent is selected from the group consisting of unsaturated 6-membered ring structures following formula (I) to (IX) 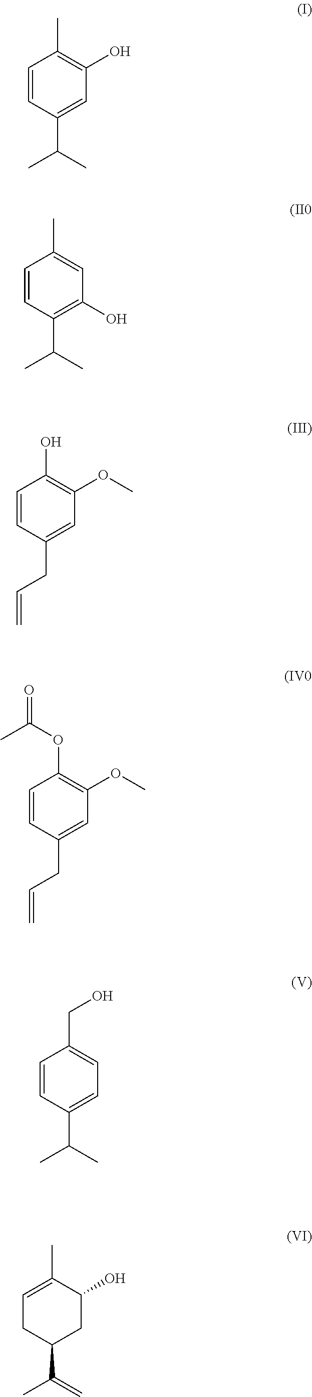 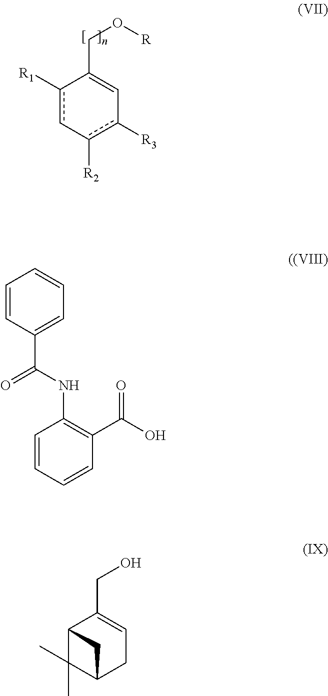
wherein R, R1, R2 and R3 independently from each other stand for hydrogen, C₁-C₃ alkyl, or C₃ alkenyl, C₁-C₃ alkoxy or acetate and n represent either zero or 1.

2. The mixture of claim 1, wherein said unsaturated six-membered ring structures are selected from the group consisting of carvacrol, thymol, eugenyl acetate, dianthramide B, eugenol, myrtenol, cumene alcohol and mixtures thereof and essential oils comprising at least one of said structures.

3. The mixture of claim 1, wherein said physiological cooling agents are selected from the group consisting of:

| Component | FEMA/GRAS |
|---|---|
| Menthol | 2665 |
| L-Menthone glyceryl ketal | 3807 |
| D,L-Menthone glyceryl ketal | 3808 |
| Menthoxy-1,2-propandiol | 3784 |
| Menthoxy-2-methyl-1,2-propanediol | 3849 |
| Menthyl ethylene glycol carbonate | 3805 |
| Menthyl formiate | 4509 |
| L-MonoMenthyl glutarate | 4006 |
| Menthyl hydroxy isobutyrate | 4308 |
| Menthyl isobutyrate | |
| L-Menthyl lactate | 3748 |
| Menthyl malonate | |
| Menthyl N-ethyl oxamate | |
| (−)-Menthyl propylene glycol carbonate | 3806 |
| (1R,2S,5R)-5-methyl-2-(propan-2-yl)-N-[2-(pyridine-2- | 4549 |
| yl)ethyl]cycloheaxecarboxamide | |
| DL-Menthyl propylene glycol carbonate | 3992 |
| Menthyl pyroglutamate | |
| Menthyl-(2-methoxy)acetate | |
| Menthyl-(2-methoxyethoxy)acetate | |
| Mono-Menthyl succinate | 3810 |
| O-Menthyl succinic acid ester amide | |
| O-Menthyl succinic acid ester-NN-(dimethyl)amide | 4230 |
| Menthane carboxylic acid-N-(4-cyanomethylphenyl)amide | 4496 |
| Menthane carboxylic acid-N-(2-(pyridine-2-yl)ethyl)amide | 4549 |
| Menthane carboxylic acid-N-ethylamide (WS-3) | 3455 |
| (WS-4) | |
| N^{α}-(menthane carbonyl) glycinic ethylester (WS-5) | 4309 |
| (1R,2S,5R)-N-(4-Methoxyphenyl)-5-methyl-2-(1-isopropyl)cyclohexane- | 4681 |
| carboxamide (WS-12) | |
| (WS-14) | |
| 2,3-dimethyl-2-(2-propyl)-butyric acid-N-methylamide (WS23) | 3804 |
| 3-(−)-Menthoxy)propane-1,2-diol | 3784 |
| Isopulegol acetate | 2965 |
| p-Menthane-3,8-diol | 4053 |
| Cubebol | 4497 |
| 3-Methyl-2(1-pyrrolidinyl)-2-cyclopentene-1-one) | |
| Tetrahydropyrimidine-2-one | |
| [(1R,2S,5R)-2-isopropyl-5-methyl-cyclohexyl] 2-(ethylamino)-2-oxo-acetate | |
| 2-(p-Tolyloxy)-N-(1H-pyrazol-5-yl)-N-((thiophen-2-yl)methyl)acetamide | 4809 |
| (E)-3-benzo[1,3]dioxol-5-yl-N,N-diphenyl-2-propenamide | 4788 |
| (E)-3-(1,3-benzodio-5-yl)-N-phenyl-N-tetrahydrofuran-3-yl-propo-2-enamide | 5002 |
| N-ethyl-2,2-diisopropylbutanamide (WS 27) | 4557 |
| 2-(4-Ethylphenoxy-N-(1H-pyrazol-5-yl)-N-((thiophen-2-yl)methyl)acetamide | 4880 |
| N-(3-hydroxy-4-methoxyphenyl)-2-isopropyl-5,5-dimethylcyclohexane-1- | 4881 |
| carboxamide | |
| N-(4-(cyanomethyl)phenyl)-2-isopropyl-5,5-dimethylcyclohexane-1- | 4882 |
| carboxamide | |

4. The mixture of claim 1, wherein the flavoring agent (a) is carvacrol and the physiological cooling agent (b) is menthyl lactate, menthyl propylene glycol carbonate, 3-(−) menthoxypropane-12-diol or mixtures thereof.

5. The mixture of claim 1, further comprising at least one aroma compound preferably wherein the aroma compound is selected from the group consisting of Anethol, Acetanisol, Acetaldehyde, Acetylmethylcarbinol, 2-Acetylpyrazine, 2-Acetylpyridine, 2-Acethylthiazoline, 2-Acethylthiazole, Allylcapronate, alpha-Amylcinnamicaldehyde, para-Anisaldehyde, Anisalcohol, Dimethylisopropyldithiazine, Benzaldehyde, Benzylacetate, l-Borneol, Butiric acid, Butylacetate, 3-Butylidenphtalide, Capric acid, l-Carvone, d-Carvone, Carvomenthone, cis-Carvylacetate, Caryophyllene, 1,8-Cineol, 1,4-Cineol, Cinnamylacetate, Citral, Citronellal, Citronellol, Citronellylacetate, Cuminaldehyde, Cyclopentadecanolide, alpha-Damascone, beta-Damascone, alpha-Damascenone, beta-Damascenone, delta-Decalactone, gamma-Decalactone, Dehydromenthofurolactone, Dihydromenthofurolactone, 2,3-Diethylpyrazine, Dihydroanethol, Dihydrocarvone, Dihydrocoumarine, beta-Dihydroionone, Dimethylanthranilate, Dimethylsulfide, Dimethylpyrazine, Sotolon, Diphenyloxide, Divanilline, 2,4-Decadienal, delta-Dodecalacton, gamma-Dodecalactone, Acetic acid, Ethylacetate, Ethylbutyrate, 2-Ethylmethylbutyrate, Ethylcapronate, Ethylcaprylate, Ethylcinnamate, Ethylisobutyrate, Ethylvanilline, Ethyllactate, Ethylmaltol, Ethylmethylthiopropionate, 4-Ethylphenol, Ethylisovalerianate, Eugenol, Fenchol, Furaneol, Filbertone, Frambinone, Frambinonmethylether, Furfurylthiol, Undecatriene, Geraniol, Geranylacetate, Geranylisobutyrate, Guaiacol, Heliotropine, 2-Heptanone, 4-Z-Heptenal, gamma-Hexalactone, gamma-Heptalactone, 3-Z-Hexenol, 2-E-Hexenol, Hexanol, Hexylacetate, 3-Z-Hexenylacetate, 2-E-Hexenylacetate, alphaHexylcinnamicaldehyde, 3-Z-Hexenylcapronate, Hotrienol, Indol, alpha-Irone, alphalonone, beta-Ionone, Isoamylacetate, Isoamylbutyrate, Isoamylisovaerianate, Isobutylacetate, Isobutylthiazol, Isobutyraldehyde, Isovaleraldehyde, Isoeugenol, Isomenthone, Isopropylmethoxypyrazine, Isobutylmethoxypyrazine, 2,4-Isopropylmethylpyrazine, Isopulegol, Jasminlactone, cis-Jasmone, Campher, Ketoisophorone, Cresol, d-Limonene, l-Linalool, d-Linalool, Linalylacetate, Linalooloxide, Maltol, Methylcyclopentenolone, L-Menthone, D-Menthone, L-Menthol, D-Menthol, Neo Menthol, L-Menthylacetate, D-Menthylacetate, Massoilactone, Melonal, 1,8-Menthenthiol, 1,8-Epithiomenthane, 8,3-Thiomenthanone, Menthofurolactone, Menthadienylacetate, 2,3-Methoxymethylpyrazine, Methylanthranilate, Methylsalicylate, Thymol, Methylbutyrate, 2-Methylbutylacetate, Methylcinnamate, 2,3-Methylfuranthiol, 2,3-Methyltetrahydrofuranthiol, Methyljasmonate, Methyldihydrojasmonate, Methylthiobutyrate, 1,3-Methylthiohexylacetate, 1,3-Methylthiohexanol, Methional, Myrtenal, Naringin, Neral, Nerol, Nerylacetate, gamma-Nonalactone, delta-Nonalactone, 2-E-Nonenal, 6-Z-Nonenal, 6-Z-Nonenol, Nootkatone, Dihydronootkatone, 1,3-Octenol, gamma-Octalactone, delta-Octalactone, Pelitorine, 1,3-Pentenone, Pentylacetate, Phenylacetaldehyde, Phenylethylalcohol, Phenylethylacetate, Piperitanat, Prenylthiol, Prenylthioacetate, Rosenoxide, Rubenamine, Rubescenamine, Sabinenhydrate, Skatole, Styrolylacetate, Terpineol, 4-Terpinenol, 1,3-Thiohexanol, 1,3-Thiohexylacetate, 2,4,4-Thiopentanone, Trimethylpyrazine, delta-Undecalactone, gamma-Undecalactone, 2,4-Decadienal, 2,4-Nonadienal, 2,6-Nonadienal, 2,4-Undecadienal, Vanilline, Vinylguaiacol, Whiskylactone, Cinnamicaldehyde, Cinnamicalcohol, Diallyldisulfide, Allylisothiocyanate, Hexanal, E-2-Hexenal, Octanal, Decanal, Tridecatrienal, 12-Methyltridecanal, alpha-Pinene, beta-Pinene, Piperitone, as well as their mixtures

6. The mixture of claim 1, further comprising at least one solvent and/or at least one aroma compound.

7. The mixture of claim 5, wherein said solvent is selected from the group consisting of water, ethanol, glycerol, 1,2-propylene glycol, 1,3-propylene glycol, triacetine, vegetable oil triglycerides and triethylcitrat and mixtures thereof preferably in a ratio 5 to about 95 wt.-percent.

8. The mixture of claim 1, wherein components (a) and (b) are present in ratios by weight of from about 10:90 to about 90:10.

9. The mixture of claim 1 being active against bacterial strains selected from the group consisting of P. gingivalis, P. intermedia and/or S. mutans.

10. The mixture of claim 1 being active against oral biofilms and/or plaque

11. An oral composition comprising a working amount of the anti-microbial mixture of claim 1.

12. The composition of claim 11, comprising the anti-microbial mixture of claim in an amount of from about 0.001 to about 1.5 wt.-percent—calculated on the composition

13. The composition of claim 11 being a toothpaste, a mouthwash, a chewing gum or a compressed tablet.

14. A method for fighting micro-organisms causing biofilms and/or plaque in the oral cavity comprising or consisting of the following steps:
(a) providing a mixture of claim 1;
(b) incorporating said mixture into an oral composition; and
(c) bringing said oral composition in contact with the oral cavity of a human.

15. The use of the mixture of claim 1 for oral care applications.
